(19) United States
(12) Patent Application Publication
(10) Pub. No.: US 2017/0216470 A1
(43) Pub. Date: Aug. 3, 2017

(54) LIGHTING DEVICE

(71) Applicants: CHIAO-AN HSIAO, Taoyuan (TW); YU-HSIU LU, Taoyuan (TW); CHUN-HUI WU, Taoyuan (TW)

(72) Inventor: YU-HSIU LU, Taoyuan (TW)

(21) Appl. No.: 15/270,173

(22) Filed: Sep. 20, 2016

(30) Foreign Application Priority Data
Feb. 1, 2016 (TW) 105201635

(51) Int. Cl. A61L 2/16; A01N 59/16; F21S 8/04

(52) U.S. Cl. CPC A61L 2/16; F21S 8/04; A01N 59/16; F21V 29/773

(57) ABSTRACT

A lighting device with a lighting function includes a coating layer coated onto the outer surface of a main body, and the coating layer is made of an antiviral material, so that the main body of the lighting device is coated with an antiviral material mainly composed of titanium dioxide and nanosilver to provide the self-cleaning and deodorizing effects, and the advantages of resisting viruses and bacteria, removing pollutants from the environment, and preventing mildews and algae effectively.

### FIELD OF THE INVENTION

The present invention relates to a lighting device, more particularly to the lighting device with a surface coated with an antiviral material, and the antiviral material mainly containing titanium dioxide and nanosilver is coated on a main body of the lighting device to achieve the stain-resistant, self-cleaning, deodorant, antivirus, and antibacterial effects, remove pollutants in the environment, and resist mildews and algae.

### BACKGROUND OF THE INVENTION

Due to global climate anomalies, pollutions resulted from over-exploitation of land, too heavy traffic, and too much construction, the quality of our living environment becomes very poor. Since people usually stay in a closed air-conditioned place to work in daytime or on street of a city fully exposed to waste gases, we always want to have a natural clean, quiet and comfortable environment to rest at nighttime. In addition, air is a necessary element of life, and the quality of air directly reflects the quality of the living environment.

In addition, the lighting device is an important tool for providing a light source indoors and at nighttime. For example, energy-saving bulbs, fluorescent lamps, downlights, stylish lamps or searchlights, etc provide a single traditional lighting function and bring the convenience of lighting the surrounding environment effectively. However, the quality of air is worsening, and floating bacteria, fungi, or microorganisms are everywhere, and the conventional lighting device does not have the function of handling the aforementioned problems. In a closed circulated air conditioning environment, the accumulation of waste gases of PM2.5 for a long time and viruses and bacteria carried by personnel passing in and out are major factors to the deterioration of the air quality and a cause to the discomfort of human body. Therefore, it is an important subject for related manufacturers to overcome the aforementioned drawbacks of the prior art by providing a lighting device with a hardware design capable of deodorizing bad smells and resisting viruses, bacteria, or fungi.

### SUMMARY OF THE INVENTION

In view of the aforementioned problems, the inventor of the present invention based on years of experience of the related industry to conduct extensively researches and experiments, and finally developed a lighting device having an antiviral material coated onto the surface of the lighting device, wherein the antiviral material coated onto the main body of the lighting device is mainly composed of titanium dioxide and nanosilver, and these materials have the advantages of self-cleaning, deodorizing, resisting viruses and bacteria, removing pollutants (such as formaldehyde) from the environment, and resisting molds and algae for the light irradiated area of the lighting device.

To achieve the aforementioned and other objectives, the present invention discloses a lighting device comprising a lamp base electrically coupled to a power supply, and a light emitting portion installed to an upper end portion of the lamp base, characterized in that a coating layer is coated onto the outer surface of the lighting device, wherein the coating layer is made of an antiviral material, and the antiviral material is composed of titanium dioxide and nanosilver.

In an embodiment of the present invention, a fastener is installed between the lamp base and the light emitting portion, and a coating layer made of an antiviral material is coated on the outer surface of the fastener.

In an embodiment of the present invention, a plurality of fins is installed to the outer surface of the lamp base, and a coating layer made of an antiviral material is coated on the outer surface of the fins.

In an embodiment of the present invention, a handle is installed to the outer surface of the lamp base, and a coating layer made of an antiviral material is coated on the outer surface of the handle.

In an embodiment of the present invention, the lamp base has a cover body covered to an outer portion of the lamp base, and a coating layer made of an antiviral material is coated on the outer surface of the cover body.

In an embodiment of the present invention, the fastener is connected to a base body, and a coating layer made of an antiviral material is coated on the outer surface of the base body.

Therefore, the lighting device of the present invention includes an antiviral material composed of titanium dioxide and nanosilver and coated onto the outer surface of the lighting device to provide the self-cleaning and deodorizing effects, and the advantages of resisting viruses and bacteria, removing pollutants (such as formaldehyde) from the environment, and preventing mildews and algae effectively for achieving an irradiate area irradiated by the light of the lighting device.

### BRIEF DESCRIPTION OF THE DRAWINGS

FIG. 1 is a perspective view of a lighting device in accordance with a first embodiment of the present invention;

FIG. 2 is a sectional view of a lighting device in accordance with the first embodiment of the present invention;

FIG. 3 is a perspective view of a lighting device in accordance with a second embodiment of the present invention;

FIG. 4 is a perspective view of a lighting device in accordance with a third embodiment of the present invention;

FIG. 5 is a perspective view of a lighting device in accordance with a fourth embodiment of the present invention;

FIG. 6 is a perspective view of a lighting device in accordance with a fifth embodiment of the present invention;

FIG. 7 is a perspective view of a lighting device in accordance with a sixth embodiment of the present invention;

FIG. 8 is a perspective view of a lighting device in accordance with a seventh embodiment of the present invention;

FIG. 9 is a perspective view of a lighting device in accordance with an eighth embodiment of the present invention;

FIG. 10 is a perspective view of a lighting device in accordance with a ninth embodiment of the present invention;

FIG. 11 is a perspective view of a lighting device in accordance with a tenth embodiment of the present invention;

FIG. 12 is a perspective view of a lighting device in accordance with an eleventh embodiment of the present invention;

FIG. 13 is a perspective view of a lighting device in accordance with a twelfth embodiment of the present invention;

FIG. 14 is a perspective view of a lighting device in accordance with a thirteenth embodiment of the present invention; and

FIG. 15 is a perspective view of a lighting device in accordance with a fourteenth embodiment of the present invention.

### DESCRIPTION OF THE PREFERRED EMBODIMENTS

To make it easier for our examiner to understand the objective of the invention, its structure, innovative features, and performance, we use a preferred embodiment together with the attached drawings for the detailed description of the invention. It is noteworthy that all figures are drawn for ease of explanation of the basic teachings of the present invention only; the extensions of the figures with respect to number, position, relationship, and dimensions of the parts to form the preferred embodiment will be explained or will be within the skill of the art after the following description has been read and understood. Further, the exact dimensions and dimensional proportions to conform to specific force, weight, strength, and similar requirements will likewise be within the skill of the art after the following description has been read and understood.

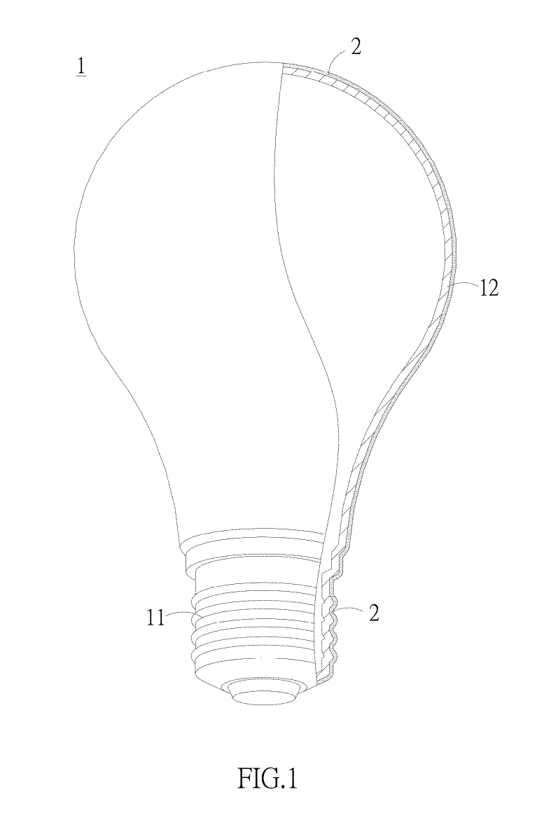

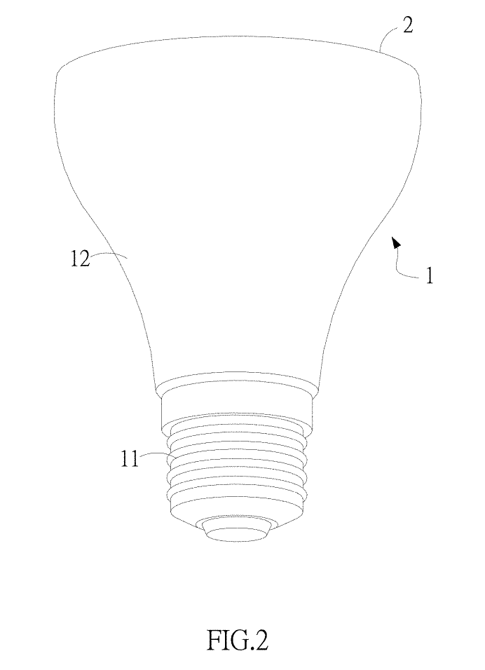

With reference to FIGS. 1 and 2 for a perspective view and a cross-sectional view of a lighting device in accordance with the first preferred embodiment of the present invention respectively, the lighting device (1) comprises a lamp base (11) electrically coupled to a power supply, and a light emitting portion (12) installed an upper end portion of to lamp base (11), characterized in that a coating layer (2) is coated onto the outer surface of the lighting device (1), wherein the coating layer (2) is made of an antiviral material, and the antiviral material is composed of titanium dioxide and nanosilver. In a preferred embodiment of the present invention, the antiviral material mainly composed of titanium dioxide and nanosilver is coated onto the outer surface of a lighting device (1) such as a bulb, so that an irradiated area irradiated by the light of the bulb has the self-cleaning and deodorizing effects, and the advantages of resisting viruses and bacteria, removing pollutants from the environment, and preventing mildews and algae effectively.

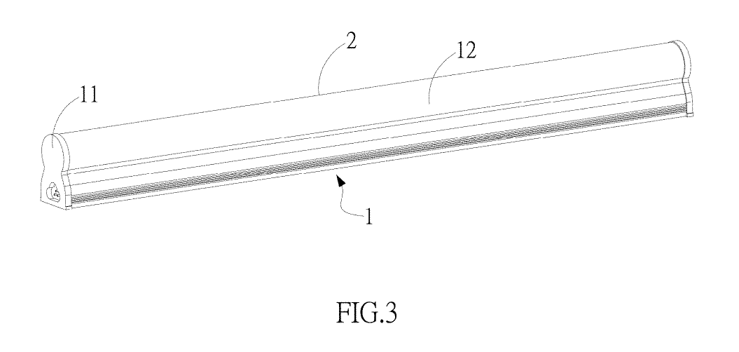

With reference to FIG. 3 for a perspective view of a lighting device in accordance with the second preferred embodiment of the present invention, the lighting device (1) is a fluorescent lamp used for area lighting, characterized in that a coating layer (2) is coated onto the outer surface of the fluorescent lamp, wherein the coating layer (2) is made of an antiviral material, and the antiviral material is mainly composed of titanium dioxide and nanosilver, so that an irradiated area irradiated by the light of the fluorescent lamp has the self-cleaning and deodorizing effects, and the advantages of resisting viruses and bacteria, removing pollutants from the environment, and preventing mildews and algae effectively.

In addition, a fastener (13) is installed between the lamp base (11) and the light emitting portion (12), and the coating layer (2) made of an antiviral material is coated onto the outer surface of the fastener (13).

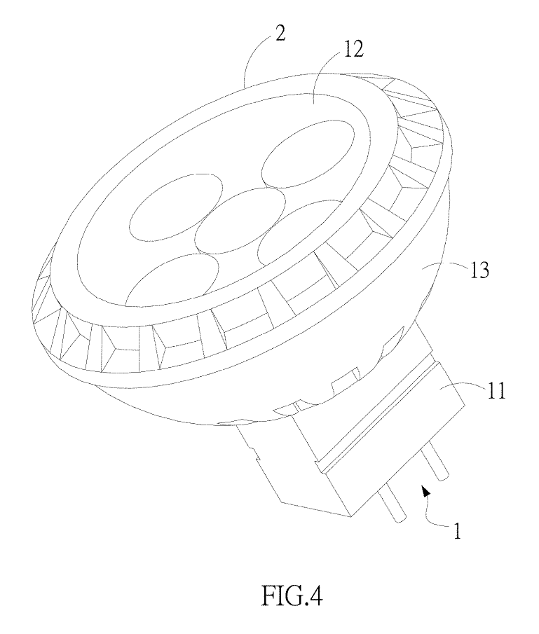

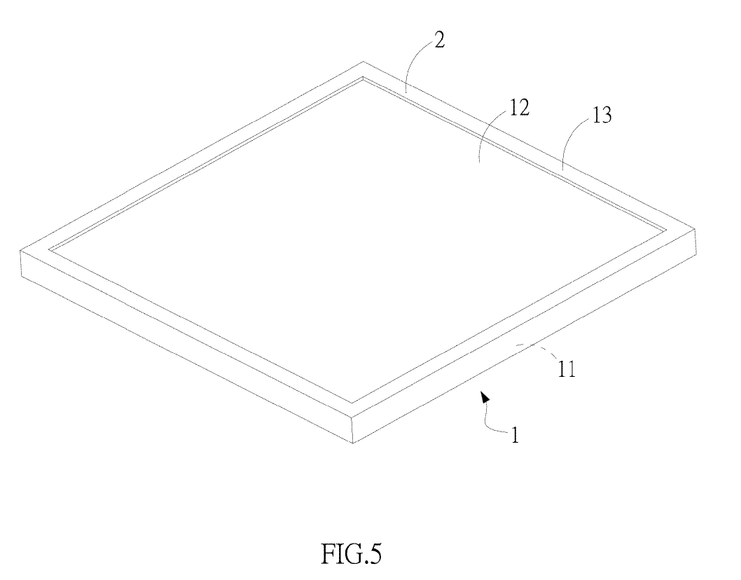

With reference to FIGS. 4 and 5 for the perspective views of the lighting devices in accordance with the third and fourth preferred embodiment of the present invention respectively, the lighting devices (1) as shown in FIGS. 4 and 5 are an energy-saving bulb and a thin flat panel light respectively used for area lighting, and a fastener (13) is installed between the lamp base (11) and the light emitting portion (12), and a coating layer (2) made of an antiviral material is coated onto the outer surface of the fastener (13), and the antiviral material mainly composed of titanium dioxide and nanosilver is coated onto the outer surface of the lighting device (1) of the energy-saving bulb or the thin flat panel light, so that an irradiated area irradiated by the light of the energy-saving bulb or the thin flat panel light has the self-cleaning and deodorizing effects, and the advantages of resisting viruses and bacteria, removing pollutants from the environment, and preventing mildews and algae effectively.

In addition, a plurality of fins (14) is installed to the outer surface of the lamp base (11), and a coating layer (2) made of an antiviral material is coated onto the outer surface of the fins (14).

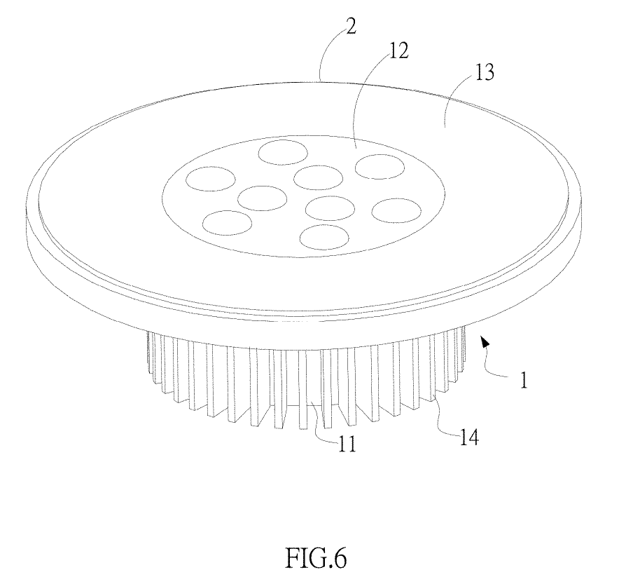

With reference to FIG. 6 for a perspective view of a lighting device in accordance with the fifth preferred embodiment of the present invention, the lighting device (1) is an energy-saving light interception type downlight, and includes a plurality of fins (14) installed to the outer surface of the lamp base (11), and a coating layer (2) made of an antiviral material is coated onto the outer surface of the fins (14), and the antiviral material mainly composed of titanium dioxide and nanosilver is coated onto the outer surface of the lighting device (1) of the energy-saving light interception type downlight, so that an irradiated area irradiated by the light of the energy-saving light interception type downlight has the self-cleaning and deodorizing effects, and the advantages of resisting viruses and bacteria, removing pollutants (such as formaldehyde) from the environment, and preventing mildews and algae effectively.

In addition, a handle (15) is installed to the outer surface of the lamp base (11), and a coating layer (2) made of an antiviral material is coated onto the outer surface of the handle (15).

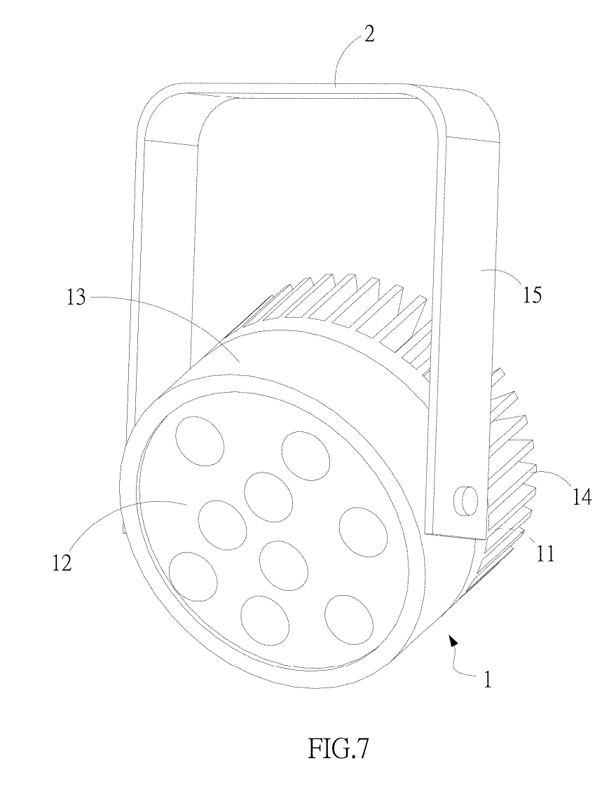

With reference to FIG. 7 for a perspective view of a lighting device in accordance with the sixth preferred embodiment of the present invention, the lighting device, the lighting device (1) is a hanging downlight used for area lighting, and a handle (15) is installed to the outer surface of the lamp base (11), and a coating layer (2) made of an antiviral material is coated onto the outer surface of the handle (15), and the antiviral material mainly composed of titanium dioxide and nanosilver is coated onto the outer surface of the lighting device (1) of the hanging downlight, so that an irradiated area irradiated by the light of the hanging downlight has the self-cleaning and deodorizing effects, and the advantages of resisting viruses and bacteria, removing pollutants (such as formaldehyde) from the environment, and preventing mildews and algae effectively.

In addition, a cover body (16) is covered onto an outer end portion of the lamp base (11), and a coating layer (2) made of an antiviral material is coated onto the outer surface of the cover body (16).

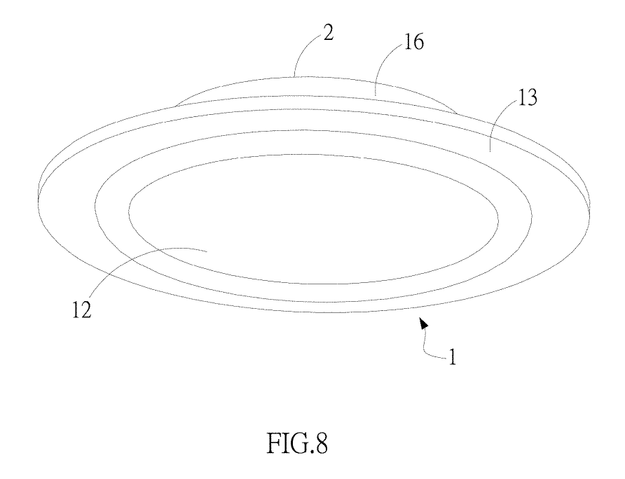

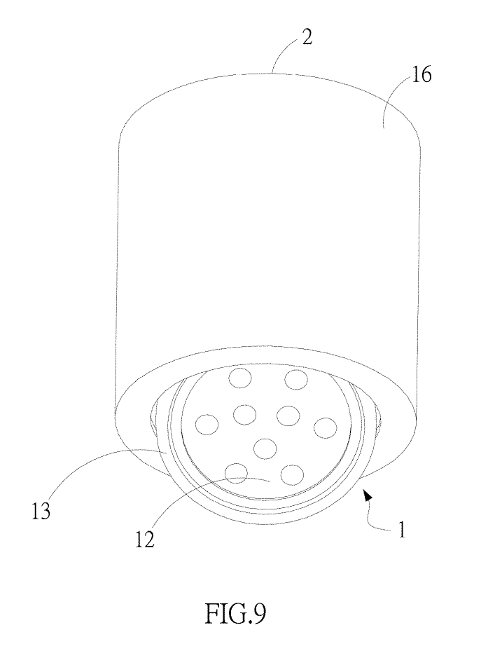

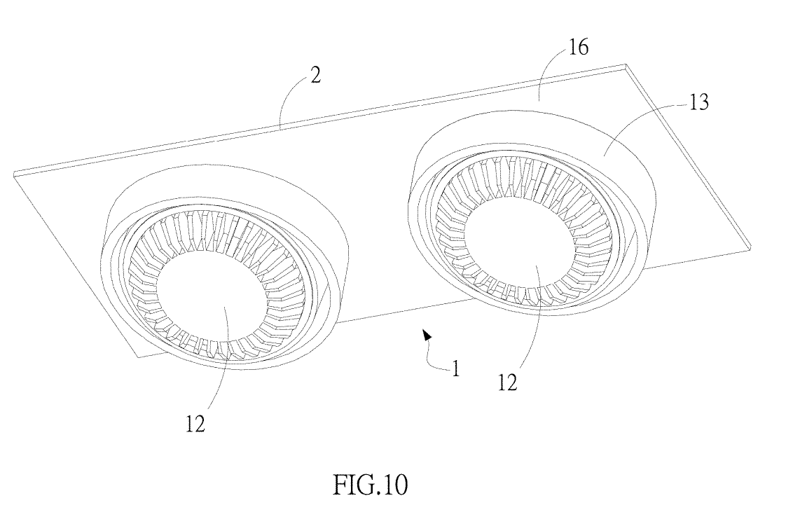

With reference to FIGS. 8-10 for the perspective views of the lighting devices in accordance with the seventh, eighth and ninth preferred embodiments of the present invention respectively, the lighting devices (1) as shown in FIGS. 8-10 are a ceiling lamp, a round downlight, and a downlight respectively used for area lighting, and a cover body (16) is covered onto an outer end portion of the lamp base (11), and a coating layer (2) made of an antiviral material is coated onto the outer surface of the cover body (16), and the antiviral material mainly composed of titanium dioxide and nanosilver is coated onto the outer surface of the lighting device (1) of the ceiling lamp, so that an irradiated area irradiated by the light of the ceiling lamp has the self-cleaning and deodorizing effects, and the advantages of resisting viruses and bacteria, removing pollutants (such as formaldehyde) from the environment, and preventing mildews and algae effectively.

In addition, a fastener (13) is coupled to a base body (17), and a coating layer (2) made of an antiviral material is coated onto the outer surface of the base body (17).

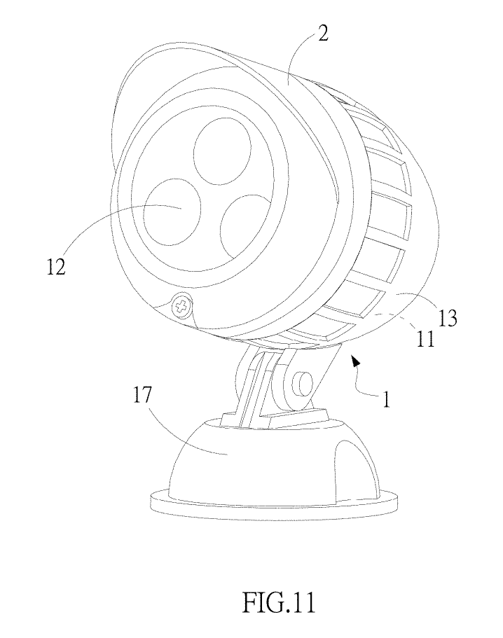

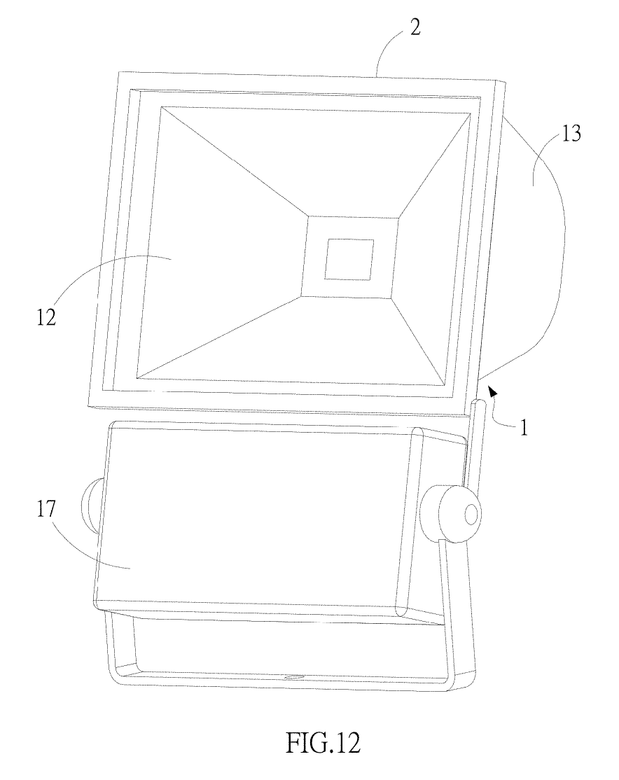

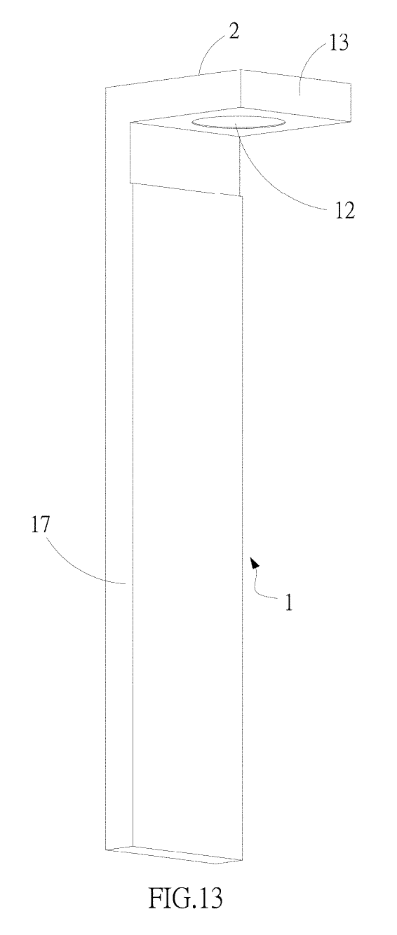

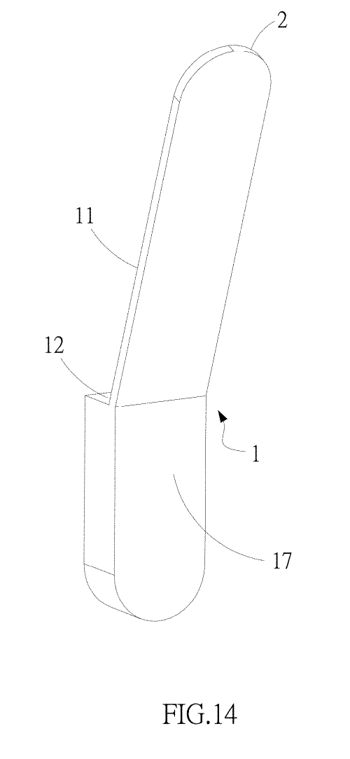

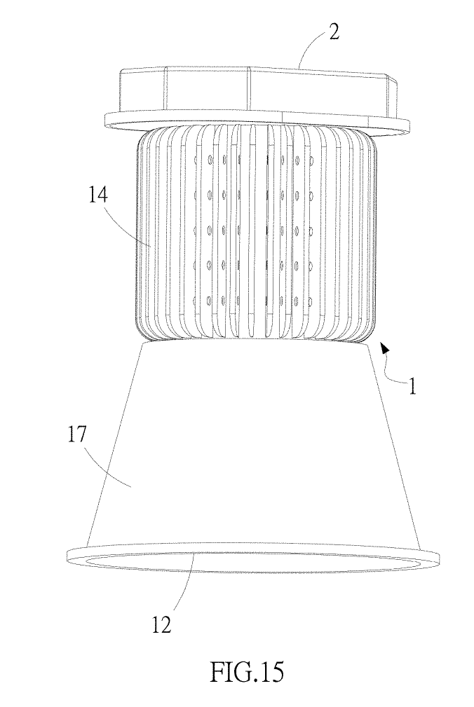

With reference to FIGS. 11-15 for the perspective views of the lighting devices in accordance with the tenth to fourteenth preferred embodiments of the present invention respectively, the lighting devices (1) are a searchlight, an outdoor lamp, a floor lamp, a stylish lamp and a specific illumination lamp respectively used for area lighting, a fastener (13) is coupled to a base body (17), and a coating layer (2) made of an antiviral material is coated onto the outer surface of the base body (17), and the antiviral material mainly composed of titanium dioxide and nanosilver is coated onto the outer surface of the lighting device (1), so that an irradiated area irradiated by the light of the lighting device (1) has the self-cleaning and deodorizing effects, and the advantages of resisting viruses and bacteria, removing pollutants (such as formaldehyde) from the environment, and preventing mildews and algae effectively.

To make it easier for our examiner to understand the objective of the invention, its structure, innovative features, and performance, we use a preferred embodiment together with the attached drawings for the detailed description of the invention. It is noteworthy that the embodiments and figures disclosed herein are to be considered illustrative rather than restrictive. When a user wants to install a lamp, the user may select the lighting device of the present invention for the self-cleaning and deodorizing effects, and the advantages of resisting viruses and bacteria, removing pollutants (such as formaldehyde) from the environment, and preventing mildews and algae effectively for the irradiated areas of the lighting device (1).

In summation, the lighting device (1) of the present invention comprises a lamp base (11) electrically coupled to a power supply and a light emitting portion (12) installed to an upper end portion of the lamp base (11), characterized in that a coating layer (2) is coated onto the outer surface of the lighting device (1), wherein the coating layer (2) is made of an antiviral material, and the antiviral material is mainly composed of titanium dioxide and nanosilver. In the present invention, the antiviral material mainly composed of titanium dioxide and nanosilver is coated onto the main body of all lighting devices (1) for the self-cleaning and deodorizing effects, and the advantages of resisting viruses and bacteria, removing pollutants (such as formaldehyde) from the environment, and preventing mildews and algae effectively for the irradiated areas of the outdoor lamps.

Compared with the prior art, the present invention has the following advantages:

1. In the lighting device of the present invention, an antiviral material composed of titanium dioxide and nanosilver is coated onto the outer surface of any lighting device, so that an irradiated area irradiated by the light of the lighting device (1) has the self-cleaning and deodorizing effects, and the advantages of resisting viruses and bacteria, removing pollutants (such as formaldehyde) from the environment, and preventing mildews and algae effectively.

In summation of the description above, the lighting device of the present invention definitely achieves the expected effect as disclosed in the embodiments, and the present invention complies with patent application requirements, and is thus duly filed for patent application.

While the invention has been described by means of specific embodiments, numerous modifications and variations could be made thereto by those skilled in the art without departing from the scope and spirit of the invention set forth in the claims.

What is claimed is:

1. A lighting device, comprising a lamp base electrically coupled to a power supply, and a light emitting portion installed to the an upper end portion of the lamp base, characterized in that a coating layer is coated onto the outer surface of the lighting device, wherein the coating layer is made of an antiviral material, and the antiviral material is composed of titanium dioxide and nanosilver.

2. The lighting device of claim 1, further comprising a fastener installed between the lamp base and the light emitting portion, and a coating layer made of an antiviral material being coated on the outer surface of the fastener.

3. The lighting device of claim 2, further comprising a plurality of fins installed to the outer surface of the lamp base, and a coating layer made of an antiviral material being coated on the outer surface of the fins.

4. The lighting device of claim 2, further comprising a handle installed to the outer surface of the lamp base, and a coating layer made of an antiviral material being coated on the outer surface of the handle.

5. The lighting device of claim 3, further comprising a handle installed to the outer surface of the lamp base, and a coating layer made of an antiviral material being coated on the outer surface of the handle.

6. The lighting device of claim 1, wherein the lamp base has a cover body covered to an outer portion of the lamp base, and a coating layer made of an antiviral material is coated on the outer surface of the cover body.

7. The lighting device of claim 2, wherein the fastener is connected to a base body, and a coating layer made of an antiviral material is coated on the outer surface of the base body.
